(19) United States
(12) Patent Application Publication
(10) Pub. No.: US 2026/0099291 A1
(43) Pub. Date: Apr. 9, 2026

(54) STREAMING SYSTEM AND STREAMING METHOD

(71) Applicant: Largan Health AI-Tech Co., Ltd., Taipei City (TW)

(72) Inventors: Cheng-Kuo LAI, Taipei City (TW); Yuan-Yen YAO, Taipei City (TW); Chen-Hsiang CHEN, Taipei City (TW); Yu-Jie HONG, Taipei City (TW)

(21) Appl. No.: 19/348,966

(22) Filed: Oct. 3, 2025

(30) Foreign Application Priority Data
Oct. 9, 2024 (TW) 113138540

(51) Int. Cl. G06F 3/14; G06F 3/16; G06F 40/143; G16H 40/67

(52) U.S. Cl. CPC G06F 3/1454; G06F 3/167; G06F 40/143; G16H 40/67

(57) ABSTRACT

A streaming system includes a measurement module, an analysis module and a display module. The measurement module includes an original data generating unit, an original data saving unit and an original data transmitting unit. The analysis module includes an original data segmenting unit, an original segmented data timestamp assigning unit, an original segmented data saving unit, an original segmented data filtering unit and a filtered segmented data processing unit. The filtered segmented data processing unit includes a filtered segmented data compressing subunit. The display module includes a data extracting unit, a filtered segmented compressed data decompressing unit, a webpage cross-platform streaming unit and a data presenting unit. The data extracting unit includes a filtered segmented compressed data extracting subunit.

### RELATED APPLICATIONS

This application claims priority to Taiwan Application Serial Number 113138540, filed Oct. 9, 2024, which is herein incorporated by reference.

### BACKGROUND

### Technical Field

The present disclosure relates to a streaming system and a streaming method. More particularly, the present disclosure relates to a streaming system and a streaming method for electrocardiograms.

### Description of Related Art

A simple measurement device includes a streamlined electrocardiogram (ECG) measurement unit, and has been widely used due to its convenience and accuracy comparable to traditional measurement instruments. A user can perform a preliminary ECG test with the simple measurement device and then view the measurement data and the analysis result through an application. However, ECG files are large and require long download times, and downloaded data also occupy the storage space of the user's device. Moreover, application compatibility varies across operating systems and devices. These circumstances reduce users' willingness to use ECG systems. To overcome the existing drawbacks and improve user acceptance, a more comprehensive ECG system needs to be developed.

### SUMMARY

According to one aspect of the present disclosure, a streaming system, which is an electrocardiogram streaming system, the streaming system includes a measurement module, an analysis module and a display module. The measurement module includes an original data generating unit, an original data saving unit and an original data transmitting unit. The analysis module is signally connected to the measurement module and the display module, and the analysis module includes an original data segmenting unit, an original segmented data timestamp assigning unit, an original segmented data saving unit, an original segmented data filtering unit and a filtered segmented data processing unit. The filtered segmented data processing unit includes a filtered segmented data compressing subunit. The display module includes a data extracting unit, a filtered segmented compressed data decompressing unit, a webpage cross-platform streaming unit and a data presenting unit. The data extracting unit includes a filtered segmented compressed data extracting subunit.

According to another aspect of the present disclosure, a streaming system, which is an electrocardiogram streaming system, the streaming system includes an analysis module and a display module. The analysis module is signally connected to the display module, and the analysis module includes an original data segmenting unit, an original segmented data timestamp assigning unit, an original segmented data saving unit, an original segmented data filtering unit and a filtered segmented data processing unit. The filtered segmented data processing unit includes a filtered segmented data compressing subunit. The display module includes a data extracting unit, a filtered segmented compressed data decompressing unit, a webpage cross-platform streaming unit, a data presenting unit and a data applying unit. The data extracting unit includes a filtered segmented compressed data extracting subunit. The data applying unit includes a data editing subunit.

According to another aspect of the present disclosure, a streaming method, which is an electrocardiogram streaming method, the streaming method includes performing a measuring step, an analyzing step and a displaying step. The measuring step includes transmitting an original data or an original compressed data. The analyzing step includes segmenting the original data into a plurality of original segmented data; adding a timestamp to each of the original segmented data; storing the original segmented data; filtering the original segmented data to generate a filtered segmented data; and processing the filtered segmented data to generate a filtered segmented compressed data. The displaying step includes extracting the filtered segmented compressed data; decompressing the filtered segmented compressed data; providing a webpage with cross-platform data streaming capability; presenting multiple data on the webpage, the data comprising the original data, the original segmented data, the filtered segmented data, or the filtered segmented compressed data; and editing the data.

### BRIEF DESCRIPTION OF THE DRAWINGS

The present disclosure can be more fully understood by reading the following detailed description of the embodiment, with reference made to the accompanying drawings as follows:

FIG. 1 is a block diagram of a streaming system according to the 1st embodiment of the present disclosure.

FIG. 2A is a block diagram of a filtered segmented data processing unit according to the 1st embodiment of FIG. 1.

FIG. 2B is a block diagram of an analysis result storing unit according to the 1st embodiment of FIG. 1.

FIG. 2C is a block diagram of a data extracting unit according to the 1st embodiment of FIG. 1.

FIG. 2D is a block diagram of a read optimizing unit according to the 1st embodiment of FIG. 1.

FIG. 2E is a block diagram of a data applying unit according to the 1st embodiment of FIG. 1.

FIG. 3 is a flow chart of a streaming method according to the 2nd embodiment of the present disclosure.

FIG. 4 is a block diagram of a streaming system according to the 3rd embodiment of the present disclosure.

FIG. 5 is a flow chart of a streaming method according to the 4th embodiment of the present disclosure.

### DETAILED DESCRIPTION

A streaming system, which is an electrocardiogram streaming system, includes a measurement module, an analysis module and a display module, the analysis module is signally connected to the measurement module and the display module. The measurement module includes an original data generating unit, an original data saving unit and an original data transmitting unit that are signal-connected. The original data generating unit is configured to obtain an original data. The analysis module includes an original data segmenting unit, an original segmented data timestamp assigning unit, an original segmented data saving unit, an original segmented data filtering unit and a filtered segmented data processing unit that are signal-connected. The original data segmenting unit is configured to segment the original data into a plurality of original segmented data. The original segmented data timestamp assigning unit is configured to add a timestamp to each of the original segmented data. The original segmented data filtering unit is configured to filter the original segmented data and generate a filtered segmented data. The filtered segmented data processing unit includes a filtered segmented data compressing subunit, which is configured to compress the filtered segmented data and generate a filtered segmented compressed data. The display module includes a data extracting unit, a filtered segmented compressed data decompressing unit, a webpage cross-platform streaming unit and a data presenting unit that are signal-connected. The data extracting unit includes a filtered segmented compressed data extracting subunit. The webpage cross-platform streaming unit is configured to provide a webpage with cross-platform data streaming capability. The data presenting unit is configured to present multiple data on the webpage, the data include an original data, an original segmented data, a filtered segmented data and a filtered segmented compressed data.

By using the webpage cross-platform streaming unit instead of downloading files, it is favorable for saving device storage space and lengthy download times so as to address the issue of large ECG files that cannot be opened immediately. The original data are segmented into multiple data by the original data segmenting unit, the capacity of each data is reduced, and it is favorable for improving the reading speed of each data. The timestamp is added to each piece of the data by the original segmented data timestamp assigning unit, and it is favorable for improving the access accuracy of each piece of the data.

The filtered segmented data compressing subunit compresses a filtered segmented data using Huffman coding. By the lossless nature of Huffman coding, it is favorable for preventing the data loss during the compression process so as to improve the accuracy of the data transmission.

A capacity of the filtered segmented compressed data of the filtered segmented compressed data extracting subunit is less than 50 KB. By controlling the capacity of the filtered segmented compressed data, it is favorable for the streaming system to balance the transmission speed and the data complexity.

The web design language of the webpage cross-platform streaming unit includes Hyper Text Markup Language 5 (HTML5). By using HTML5 as the web design language, it is favorable for increasing the compatibility of webpages with various operating systems and browsers so as to improve the versatility of the streaming system.

The display module can further include a webpage time limiting unit, the webpage time limiting unit is signally connected to the data extracting unit, the filtered segmented compressed data decompressing unit, the webpage cross-platform streaming unit and the data presenting unit. The webpage time limiting unit is configured to set a limited time for the webpage. The URL's access rights to the webpage can be limited in time by the webpage time limiting unit, and it is favorable for preventing the data from being viewed indefinitely so as to enhance privacy.

The display module can further include a data temporary saving unit, the data temporary saving unit is signally connected to the data extracting unit, the filtered segmented compressed data decompressing unit, the webpage cross-platform streaming unit, the data presenting unit and the webpage time limiting unit. The data temporary saving unit is configured to store the data temporarily in the memory of the user device. Through the data temporary saving unit, when the user device reads the data, the streaming system does not need to repeatedly provide the data to the user, and it is favorable for reducing the system load of the streaming system.

The display module can further include a data applying unit, and the data applying unit is signally connected to the data extracting unit, the filtered segmented compressed data decompressing unit, the webpage cross-platform streaming unit, the data presenting unit, the webpage time limiting unit and the data temporary saving unit. The data applying unit includes a data editing subunit, and the data editing subunit is configured to edit the data. Through the data editing subunit, it is favorable for manager to modify the data and analyze results so as to improve the data accuracy.

The display module can further include an URL standardization unit, the URL standardization unit is signally connected to the data extraction unit, the filtered segmented compressed data decompressing unit, the webpage cross-platform streaming unit, the data presentation unit, the webpage time limiting unit, the data temporary saving unit and the data applying unit. The URL standardization unit is configured to make the domain name or the path name of the webpage fixed or partially fixed. Complicated URL is arranged in a regularized manner for easier memorization by the URL standardization unit, and it is favorable increasing the user willingness to use the streaming system.

The display module can further include a read optimizing unit, the read optimizing unit is signally connected to the data extracting unit, the filtered segmented compressed data decompressing unit, the webpage cross-platform streaming unit, the data presenting unit, the webpage time limiting unit, the data temporary saving unit and the data applying unit. The read optimizing unit includes a memory setting subunit, and the memory setting subunit is configured to set the maximum usage of the memory of the user device. The user can customize the maximum memory capacity of the streaming system by the memory setting subunit, and it is favorable for utilizing the user device resources appropriately so as to prevent the user device from running out of memory due to excessive temporary data.

The read optimizing unit can further include a data reading speed setting subunit, and the data reading speed setting subunit is configured to set the reading speed for the user device. The data reading speed of the user device is limited by the data reading speed setting subunit, and it is favorable for reducing the network load of the user device.

The read optimizing unit can further include a data reading speed displaying subunit, and the data reading speed displaying subunit is configured to display the reading speed of the user device. The reading speed of the user device is displayed by the data reading speed displaying subunit, and it is favorable for the user to quickly understand the network load of the user device.

The read optimizing unit can further include a data pre-fetching subunit, and the data pre-fetching subunit is configured to allow the user device to pre-fetch the data in both the negative time axes and the positive time axes relative to the current timestamp. The data that the user is likely to view can be retrieved in advance by the data pre-fetching subunit, and the user does not need to load the data in real time while viewing, and it is favorable for reducing the latency of the streaming system.

The data applying unit can further include a data comparing subunit, and the data comparing subunit is configured to compare the data. The data from different subjects are compared rapidly by the data comparing subunit, and it is favorable for the user to quickly understand the data differences.

The data applying unit can further include an edited record displaying subunit, and the edited record displaying subunit is configured to display an edit record for each piece of the data. The edit record of the data is displayed by the edited record displaying subunit, and it is favorable for tracking the data edits.

The data applying unit can further include a data sharing subunit, and the data sharing subunit is configured to share the data. The data are shared by the data sharing subunit, and it is favorable for the circulation of the data.

The data applying unit can further include a data section downloading subunit, and the data section downloading subunit is configured to select a start time and an end time of the data when downloading the data. The specific time interval for downloading the data is selected by the data section downloading subunit, and it is favorable for the user to view the data offline and reducing the storage of useless data.

The display module can further include a facilitation unit, and the facilitation unit includes an interface customization subunit. The interface customization subunit is configured to modify an interface of the streaming system. The user can modify the interface by the interface customization subunit, and it is favorable for improving the practicality of the interface.

The facilitation unit can further include a voice subunit, and the voice subunit is configured to operate the streaming system according to a voice command. The user can quickly give instructions through language by the voice subunit, and it is favorable for improving operational convenience.

A streaming method, which is an electrocardiogram streaming method, the streaming method includes performing a measuring step, an analyzing step and a displaying step. The measuring step includes transmitting an original data or an original compressed data. The analyzing step includes segmenting the original data into a plurality of original segmented data; adding a timestamp to each of the original segmented data; storing the original segmented data; filtering the original segmented data to generate a filtered segmented data; and processing the filtered segmented data to generate a filtered segmented compressed data. The displaying step includes extracting the filtered segmented compressed data; decompressing the filtered segmented compressed data; providing a webpage with cross-platform data streaming capability; presenting multiple data on the webpage, the data comprising the original data, the original segmented data, the filtered segmented data, or the filtered segmented compressed data; and editing the data. By using the webpage cross-platform streaming instead of downloading files, it is favorable for saving device storage space and lengthy download times so as to address the issue of large ECG files that cannot be opened immediately. The capacity of each data is reduced by segmenting the original data into multiple data, and it is favorable for improving the reading speed of each data. By adding the timestamp to each piece of the data, it is favorable for improving the access accuracy of each piece of the data. By editing the data, it is favorable for managers to correct the data and analyze results so as to improve the data accuracy.

Each of the aforementioned features of the streaming system can be utilized in various combinations for achieving the corresponding effects.

The streaming of the present disclosure refers to a process in which the user can view the data while the data is being retrieved or read, and the data is stored in the temporary storage unit rather than a storage unit. The temporary storage unit can be a random access memory (RAM), and the storage unit can be a disk, but the present disclosure is not limited thereto.

The streaming system of the present disclosure can be an electrocardiogram streaming system, a respiratory rhythm graph streaming system, or a sleep cycle graph streaming system. The streaming system can include at least one module, at least two modules, at least three modules, at least four modules, or at least five modules, wherein the streaming system can include the measurement module, the analysis module and the display module. The streaming system can include at least one measurement module, at least two measurement modules, at least three measurement modules, at least four measurement modules, or at least five measurement modules. The streaming system can include at least one analysis module, at least two analysis modules, at least three analysis modules, at least four analysis modules, or at least five analysis modules. The streaming system can include at least one display module, at least two display modules, at least three display modules, at least four display modules, or at least five display modules. The location of the streaming system is determined based on the locations of the entities of each module and the users. The entities of each module of the streaming system can be located in the same domain, different domains, the same location, different locations, the same country, or different countries. The users of each module of the streaming system can be located in the same country or different countries. When the entities of each module of the streaming system and the user are located in different countries, the streaming system can be considered to be located in the countries where the entities of each module and the users are located. For example, when the entity of the measurement module is located in the United States, the user of the measurement module is located in Japan, the entity of the analysis module is located in the Netherlands, the user of the analysis module is located in France, the entity of the display module is located in Australia, and the user of the display module is located in South Korea, then the streaming system is located in the United States, Japan, the Netherlands, France, Australia, and South Korea at the same time.

The electrocardiogram (ECG) of the present disclosure refers to an image produced by electrocardiography, which records the electrical activity of the heart. An ECG typically includes P waves, Q waves, R waves, S waves, and T waves, the axes of the ECG typically include time and voltage. ECG analysis involves interpreting the timing and voltage of the P, Q, R, S, and T waves.

The module of the present disclosure can be an electronic component, unit, device, instrument, host or server, etc., but this disclosure is not limited thereto.

The units of each module of the present disclosure can include an original data generation function, an original data saving function, an original data transmission function, an original data compression function, an original compressed data transmission function, an original compressed data decompression function, an original data segmentation function, an original segmented data timestamp assignment function, an original segmented data saving function, an original segmented data filtering function, a filtered segmented data processing function, an analysis result storage function, a data extraction function, a filtered segmented compressed data decompression function, a webpage cross-platform streaming function, a webpage time limiting function, a read optimization function, a data temporary saving function, a data presentation function, a data application function, a URL standardization function and a facilitation function.

The streaming method of the present disclosure can be an electrocardiogram streaming method, a respiratory rhythm graph streaming method, or a sleep cycle graph streaming method. The streaming method can include at least one step, at least two steps, or at least three steps, wherein each step of the streaming method can include the measuring step, the analyzing step, and the displaying step. Each execution action of each step of the streaming method is based on each unit of each module of the streaming system, and each execution action is the description of the operation of each module and each unit. The location of the streaming method is determined by the location of the devices and users at each step. The devices at each step of the streaming method can be located in the same country or in different countries, and the users at each step of the streaming method can be located in the same country or in different countries. When the devices and the users of each step of a streaming method are located in different countries, the streaming method can be considered to be applied simultaneously to the countries in which each device and user resides. For example, if the device used in the measuring step is located in the United States, the user of the measuring step is located in Japan, the device used in the analyzing step is located in the Netherlands, the user of the analyzing step is located in France, and the device used in the displaying step is located in Australia, and the user of the displaying step is located in South Korea, then the streaming method can be applied simultaneously to the United States, Japan, the Netherlands, France, Australia, and South Korea.

The execution actions of the present disclosure can include original data generation, original data saving, original data compression, original compressed data transmission, original compressed data decompression, original data segmentation, timestamp assignment for segmented original data, original segmented data saving, original segmented data filtering, filtered segmented data processing, analysis result storage, data extraction, filtered segmented compressed data decompression, cross-platform webpage streaming, webpage time limitation, read optimization, temporary data saving, data presentation, data application, URL standardization, and facilitation. The respective steps can sequentially include original data generation, original data saving, original data transmission or original compressed data transmission, original data segmentation, timestamp assignment for segmented original data, original segmented data saving, original segmented data filtering, filtered segmented data processing, analysis result storage, data extraction, filtered segmented compressed data decompression, cross-platform webpage streaming, webpage time limitation, read optimization, temporary data saving, data presentation, and data application.

The original data described of the present disclosure refers to the data directly output after measurement and not processed by noise suppression, noise elimination, waveform correction, or other processing methods.

The raw data generating unit of the present disclosure has the capability of generating the original data through measurement.

The original data saving unit of the present disclosure has the capability of saving the original data.

The original data compression unit of the present disclosure has the capability for compressing the original data. The aforementioned compression method can be Arithmetic coding, Asymmetric Numeric System (ANS) coding, Golomb coding, Huffman coding, Interval coding, Shannon coding, Shannon-Fano coding, Shannon-Fano-Elias coding, Tunstall coding, Unary coding, Exponential-Golomb coding, Fibonacci coding, Elias gamma coding, or Levenshtein coding.

The original data transmitting unit of the present disclosure has the capability of actively transmitting the original data.

The original compressed data of the present disclosure refers to the data obtained by compressing the original data.

The original compressed data transmitting unit of the present disclosure has the capability of actively transmitting the original compressed data.

The original compressed data decompressing unit of the present disclosure has the capability of decompressing the original compressed data.

The original data segmenting unit of the present disclosure has the capability of segmenting the original data into multiple pieces of the original segmented data.

The original segmented data of the present disclosure refers to the data obtained by segmenting the original data.

The original segmented data timestamp assigning unit of the present disclosure has the capability of assigning the timestamp to the original segmented data as a time mark.

The original segmented data saving unit of the present disclosure has the capability of storing the original segmented data in the storage unit.

The original segmented data filtering unit of the present disclosure has the capability of filtering the original segmented data. Filtering refers to processing such as noise suppression, noise elimination, and waveform correction on the data.

The filtered segmented data described of the present disclosure refers to the data obtained by filtering the original segmented data.

The filtered segmented data processing unit of the present disclosure has the capability of processing the filtered segmented data. The filtered segmented data processing unit can include a filtered segmented data analysis subunit and/or a filtered segmented data compressing subunit.

The filtered segmented data analysis subunit of the present disclosure has the capability of analyzing the filtered segmented data.

The filtered segmented data compressing subunit of the present disclosure has the capability of compressing the filtered segmented data.

The analysis result of the present disclosure refers to the judgment result obtained by analyzing the original data, the original segmented data, or the filtered segmented data. The analysis result is usually the judgment of various physiological states. The analysis result can be used to judge various physiological states by simple labels, such as: Y or V represents abnormality, and N represents no abnormality. The aforementioned physiological states that can be judged include hypercalcemia, hypocalcemia, dysrhythmia, tachycardia, bradycardia, arrhythmia, conduction block, accelerated conduction, cardiac ischemia, myocardial hypoxia, myocardial infarction, atrial fibrillation, ventricular fibrillation, atrial hypertrophy, ventricular hypertrophy, coronary artery ischemia, etc.

The analysis result storing unit of the present disclosure refers to the capability of storing the analysis result. The analysis result storing unit can include an analysis result saving subunit and/or an analysis result temporary saving subunit.

The analysis result saving subunit of the present disclosure has the capability of saving the analysis result. Saving refers to storing the analysis result in a storage unit.

The analysis result temporary saving subunit of the present disclosure has the capability of temporarily saving the analysis result. Temporarily saving refers to storing the analysis result in the temporary storage unit.

The data of the present disclosure can include the original data, the original segmented data, the filtered segmented data, the filtered segmented compressed data and the analysis result.

The data extraction unit described of the present disclosure has the capability of extracting the data from the module. The data extraction unit can include an analysis result extracting subunit and/or a filtered segmented compressed data extracting subunit.

The analysis result extracting subunit of the present disclosure has the capability of extracting the analysis result.

The filtered segmented compressed data of the present disclosure refers to the data obtained by compressing the filtered segmented data. The smaller the capacity of the filtered segmented compressed data, the faster the transmission of related steps and functions. Controlling the capacity of the filtered segmented compressed data can balance transmission speed and the data complexity. The capacity of the filtered segmented compressed data can be less than 500 KB, less than 400 KB, less than 300 KB, less than 200 KB, less than 100 KB, less than 80 KB, less than 50 KB, less than 30 KB, less than 28 KB, less than 25 KB, less than 22 KB, or less than 20 KB. The capacity of the filtered segmented compressed data can be greater than 1 KB, greater than 3 KB, greater than 5 KB, greater than 8 KB, greater than 10 KB, greater than 13 KB, greater than 15 KB, greater than 17 KB, or greater than 19 KB. The capacity of the filtered segmented compressed data mentioned above refers to the average capacity, and the capacity of the filtered segmented compressed data must be calculated by averaging at least 10 consecutive filtered segmented compressed data.

The filtered segmented compressed data extracting subunit of the present disclosure has the capability of extracting the filtered segmented compressed data.

The filtered segmented compressed data decompression unit of the present disclosure has the capability of decompressing filtered segmented compressed data.

The webpage cross-platform streaming unit of the present disclosure has the capability of making a webpage cross-platform and streaming. The webpage cross-platform streaming unit can span operating systems such as Mac OS, Windows, iOS, Android, and Linux. The webpage design language of the webpage cross-platform streaming unit can include XML, HTML, CSS, Javascript, PHP, GenCode, TeX, SGML, RDF, XForms, DocBook, SOAP, WOL, wherein HTML can be HTML5, but the present disclosure is not limited thereto.

The webpage time limiting unit of the present disclosure has the capability of setting the time limit for the webpage. When the time limit is exceeded, the webpage will not display the data.

The read optimizing unit of the present disclosure has the capability of improving the reading speed or reading fluency of the user device. The read optimizing unit can include a memory setting subunit, a data reading speed setting subunit, a data reading speed displaying subunit and a data pre-fetching subunit.

The memory setting subunit described of the present disclosure has the capability of providing the user with a customizable maximum memory usage of the display module for the user device. When the memory usage of the display module for the user device reaches the maximum memory usage, the display module will automatically delete the temporary data occupying the memory of the user device. The temporary data automatically deleted may be the earliest read temporary data, or the temporary data with the furthest time from the timestamp of the currently viewed data. For example, the maximum memory usage can be less than or equal to 500 MB, 1 GB, 1.5 GB, 2 GB, 2.5 GB, 3 GB, 3.5 GB, and 4 GB; the maximum memory usage can be greater than or equal to 10 MB, 20 MB, 30 MB, 40 MB, 50 MB, 80 MB, 100 MB, 120 MB, 150 MB, 200 MB, 250 MB, 280 MB, and 300 MB.

The data reading of the present disclosure refers to the reading of the data by the user device.

The data reading speed setting subunit of the present disclosure has the capability of allowing the user to customize the data reading speed of the user device.

The data reading speed displaying subunit of the present disclosure has the capability of displaying the current data reading speed of the user device.

The data pre-fetching subunit of the present disclosure has the capability of allowing the user device to pre-fetch data on the negative and positive time axes of the current timestamp when the user is viewing the data.

The data temporary saving unit of the present disclosure has the capability of temporarily storing the data in the user device memory when the user device is reading the data.

The data presenting unit of the present disclosure has the capability of presenting the data to the user device.

The data applying unit of the present disclosure has the capability of applying various data applications. The data applying unit can include a data comparing subunit, a data editing subunit, an edited record displaying subunit, a data sharing subunit and a data section downloading subunit.

The data comparing subunit of the present disclosure has the capability of comparing multiple data. The data comparison can be performed side by side or in an overlay. The multiple data can be the data at different time points, the data from different subjects, and the standard data. The standard data refers to the patient data or the data from healthy individuals that can be used as a comparison standard.

The data editing subunit of the present disclosure has the capability of providing the user device with the ability to edit the data. The data editing subunit includes functions such as data modification, data deletion, multiple additions of analysis result, analysis result modification, analysis result deletion, providing suggestions, and marking annotations. The user of the aforementioned data editing subunit is typically the management personnel, who may be a streaming system maintenance personnel or medical personnel.

The edited record display subunit of the present disclosure has the capability of providing the user device with the ability to display the data editing records. The data editing records can include the editor, editing time and editing content.

The data sharing subunit of the present disclosure has the capability of sharing the data with others. The data sharing subunit can be achieved by transmitting a specific URL, granting specific webpage permissions, etc.

The data section downloading subunit of the present disclosure has the capability of selecting the start time and the end time of the data when downloading the data to the storage unit of the user device. The aforementioned data can include the original data, the original segmented data, the filtered segmented data and the analysis result.

The URL standardization unit of the present disclosure has the capability of making the domain name or path name of the URL where each piece of the data is located fixed or partially fixed when the URL is used to direct the webpage where the data is located. For example, the domain name of the URL is fixed, or the path name of the URL is partially fixed.

The facilitation unit of the present disclosure has the capability of improving the user operation convenience. The facilitation unit can include an interface customization subunit and a voice subunit. The displaying step can include facilitation, which can be used in any order of the displaying steps. Facilitation can be performed before webpage time-limiting, before loading optimization, before data temporary storage, before data presentation, or before data application. Facilitation can be performed after webpage cross-platform streaming, after webpage time-limiting, after loading optimization, after data temporary storage, or after data presentation.

The interface customization subunit of the present disclosure allows the user to modify the system interface according to their preferences or needs. The interface customization subunit primarily modifies the visual complexity of the interface, allowing the system interface to modify various unit buttons. For example, the user can hide, display, enlarge, reduce, change the color, or change the shape of page backgrounds, function buttons and icons.

The voice subunit of the present disclosure allows the user to give commands to the system using voice. The commands that can be given by the voice subunit include searching, using various functions, and selecting data time. The aforementioned use of various functions refers to the use of the functions of each module.

According to the above embodiment, specific examples are proposed below and explained in detail with the drawings.

### 1st Embodiment

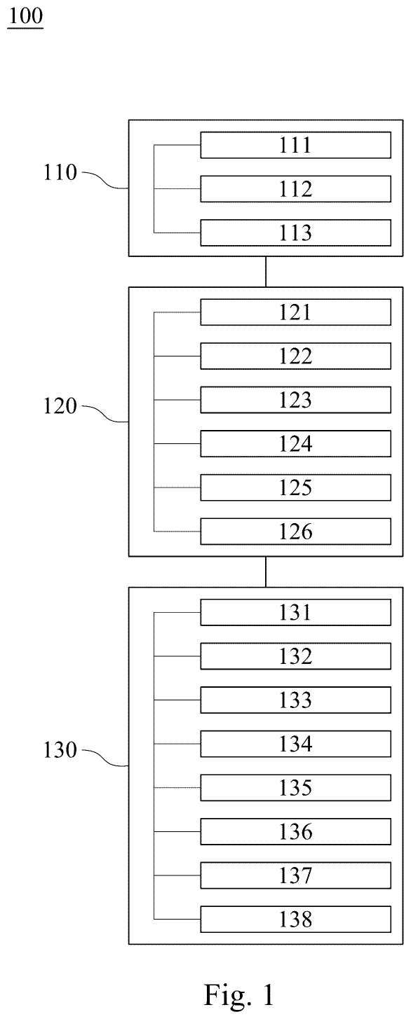

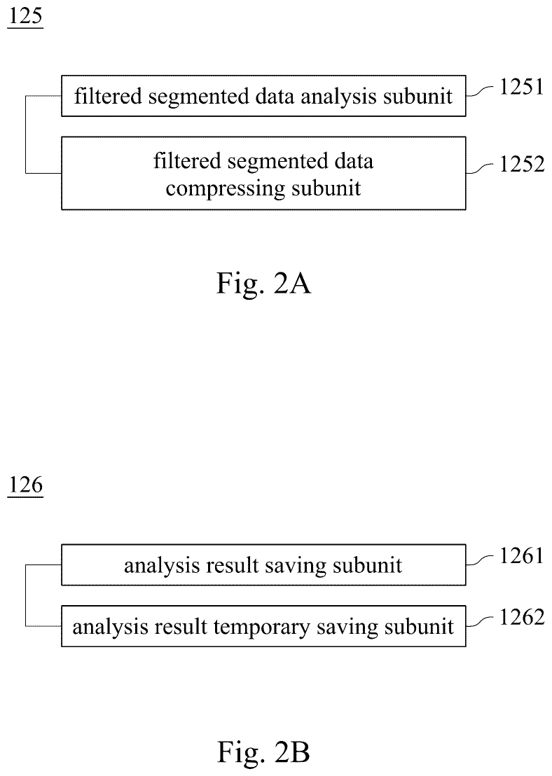

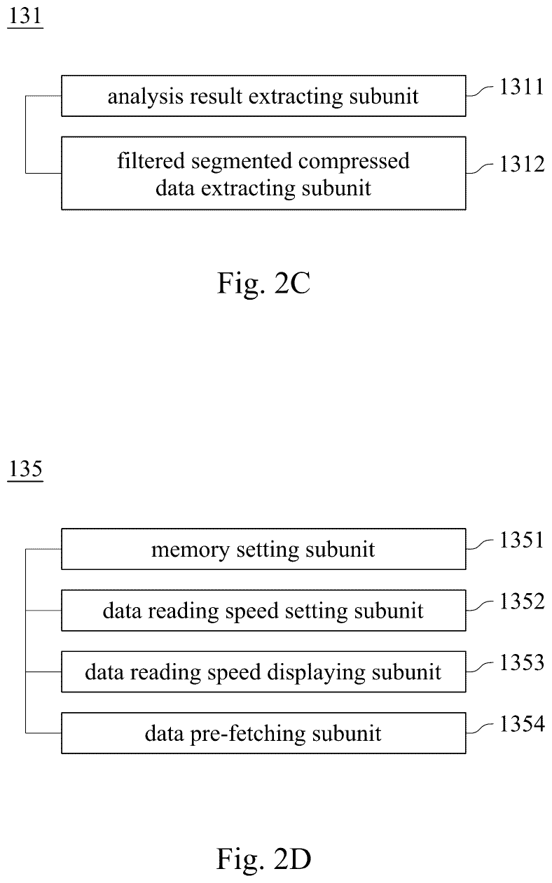

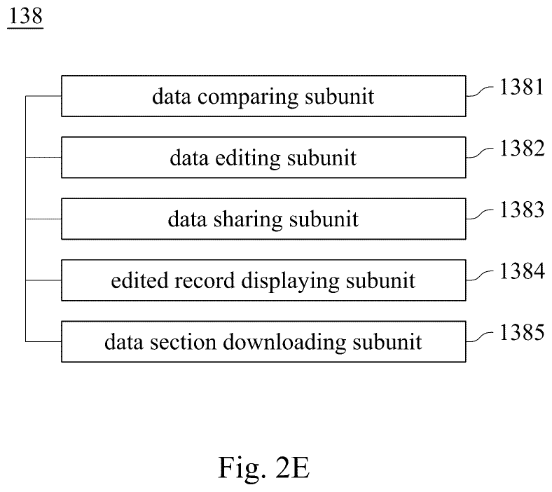

FIG. 1 is a block diagram of a streaming system 100 according to the 1st embodiment of the present disclosure. FIG. 2A is a block diagram of a filtered segmented data processing unit 125 according to the 1st embodiment of FIG. 1. FIG. 2B is a block diagram of an analysis result storing unit 126 according to the 1st embodiment of FIG. 1. FIG. 2C is a block diagram of a data extracting unit 131 according to the 1st embodiment of FIG. 1. FIG. 2D is a block diagram of a read optimizing unit 135 according to the 1st embodiment of FIG. 1. FIG. 2E is a block diagram of a data applying unit 138 according to the 1st embodiment of FIG. 1. As shown in FIG. 1, FIG. 2A, FIG. 2B, FIG. 2C, FIG. 2D and FIG. 2E, in the 1st embodiment, the streaming system 100 is the electrocardiogram streaming system.

The streaming system 100 includes a measurement module 110, an analysis module 120 and a display module 130, the analysis module 120 is signally connected to the measurement module 110 and the display module 130. The measurement module 110 includes an original data generating unit 111, an original data saving unit 112 and an original data transmitting unit 113 that are signal-connected. The original data generating unit 111 is configured to obtain an original data from cardiac measurement. The original data saving unit 112 is configured to save the original data. The original data transmitting unit 113 is configured to transmit the original data to the analysis module 120.

The analysis module 120 includes an original data segmenting unit 121, an original segmented data timestamp assigning unit 122, an original segmented data saving unit 123, an original segmented data filtering unit 124, a filtered segmented data processing unit 125, and an analysis result storing unit 126 that are signal-connected. The original data segmenting unit 121 is configured to segment the original data into a plurality of original segmented data. The original segmented data timestamp assigning unit 122 is configured to add a timestamp to each of the original segmented data. The original segmented data saving unit 123 is configured to save the original segmented data. The original segmented data filtering unit 124 is configured to filter the original segmented data and generate a filtered segmented data.

As shown in FIG. 2A, the filtered segmented data processing unit 125 includes a filtered segmented data analysis subunit 1251 and a filtered segmented data compressing subunit 1252 that are signal-connected. The filtered segmented data analysis subunit 1251 is configured to analyze the filtered segmented data. The filtered segmented data compression subunit 1252 is configured to compress the filtered segmented data and generate a filtered segmented compressed data. As shown in FIG. 2B, the analysis result storing unit 126 includes an analysis result saving subunit 1261 and an analysis result temporary saving subunit 1262 that are signal-connected. The analysis result saving subunit 1261 is configured to save the analysis result. The analysis result temporary saving subunit 1262 is configured temporarily saving the analysis result. The analysis result include judgment results obtained by analyzing the original data, the original segmented data and the filtered segmented data, typically for various physiological conditions. The analysis result can be simply labeled as the various physiological conditions.

The display module 130 includes a data extracting unit 131, a filtered segmented compressed data decompressing unit 132, a webpage cross-platform streaming unit 133, a webpage time limiting unit 134, read optimizing unit 135, a data temporary saving unit 136, a data presenting unit 137 and a data applying unit 138 that are signal-connected. As shown in FIG. 2C, the data extracting unit 131 includes an analysis result extracting subunit 1311 and a filtered segmented compressed data extracting subunit 1312 that are signal-connected. The analysis result extracting subunit 1311 is configured to extract the analysis result. The filtered segmented compressed data extracting subunit 1312 is configured to extract the filtered segmented compressed data. The filtered segmented compressed data decompressing unit 132 is configured to decompress the filtered segmented compressed data. The webpage cross-platform streaming unit 133 is configured to provide the webpage with the cross-platform data streaming capability. The webpage time limiting unit 134 is configured to set a limited time for the webpage.

As shown in FIG. 2D, the read optimizing unit 135 includes a memory setting subunit 1351, a data reading speed setting subunit 1352, a data reading speed displaying subunit 1353 and a data pre-fetching subunit 1354 that are signal-connected. The memory setting subunit 1351 is configured to set the maximum usage of the memory of the user device. The data reading speed setting subunit 1352 is configured to set the reading speed for the user device. The data reading speed displaying subunit 1353 is configured to display the reading speed of the user device. The data pre-fetching subunit 1354 is configured to allow the user device to pre-fetch the data in both the negative time axes and the positive time axes relative to the current timestamp. The data temporary saving unit 136 is configured to store the data temporarily in the memory of the user device when the user device reads the data. The data presenting unit 137 is configured to present multiple data on the webpage, the data include the original data, the original segmented data, the filtered segmented data, the filtered segmented compressed data and the analysis result. As shown in FIG. 2E, the data applying unit 138 includes a data comparing subunit 1381, a data editing subunit 1382, a data sharing subunit 1383, an edited record displaying subunit 1384 and a data section downloading subunit 1385. The data comparing subunit 1381 is configured to compare the data. The data editing subunit 1382 is configured to edit the data. The data sharing subunit 1383 is configured to share the data. The edited record displaying subunit 1384 is configured to display an edit record for each piece of the data. The data section downloading subunit 1385 is configured to select a start time and an end time of the data when downloading the data.

Thus, the original data are segmented into multiple data by the original data segmenting unit 121, the capacity of each data is reduced, and it is favorable for improving the reading speed of each data. The timestamp is added to each piece of the data by the original segmented data timestamp assigning unit 122, and it is favorable for improving the access accuracy of each piece of the data. By using the webpage cross-platform streaming unit 133 instead of downloading files, it is favorable for saving device storage space and lengthy download times so as to address the issue of large ECG files that cannot be opened immediately.

### 2nd Embodiment

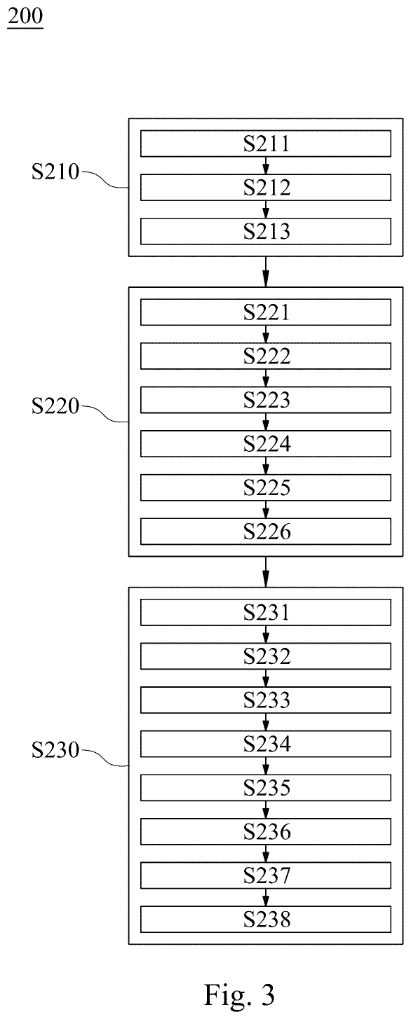

FIG. 3 is a flow chart of a streaming method 200 according to the 2nd embodiment of the present disclosure. The streaming method 200 includes performing a measuring step S210, an analyzing step S220 and a displaying step S230. As shown in FIG. 3, in the 2nd embodiment, the streaming method 200 is the electrocardiogram streaming method. The measuring step S210 includes step S211, step S212 and step S213. Step S211 includes measuring the heart to generate the original data. Step S212 includes saving the original data. Step S213 includes transmitting the original data.

The analyzing step S220 includes step S221, step S222, step S223, step S224, step S225 and step S226. Step S221 includes segmenting the original data into a plurality of original segmented data. Step S222 includes adding the timestamp to each original segmented data. Step S223 includes saving the original segmented data. Step S224 includes filtering the original segmented data to generate a filtered segmented data. Step S225 includes analyzing and compressing the filtered segmented data to generate filtered segmented compressed data. Step S226 includes saving the analysis result.

The displaying step S230 includes step S231, step S232, step S233, step S234, step S235, step S236, step S237 and step S238. Step S231 includes extracting the analysis result. Step S232 includes decompressing the filtered segmented compressed data. Step S233 includes providing the webpage with cross-platform data streaming capability. Step S234 includes setting the limited time for the webpage. Step S235 includes optimizing webpage access to further set the maximum memory usage and reading speed of the user device. Step S236 includes storing the data temporarily in the user device's memory while the user device is accessing the data. Step S237 includes presenting multiple data on the webpage. Step S238 includes providing the user with the ability to compare, edit, share, and download the data on the user device.

In detail, the streaming system 100 of the 1st embodiment of FIG. 1 is configured to implement the streaming method 200 of the 2nd embodiment, and is configured to replace downloading files through the webpage cross-platform streaming. It is favorable for saving device storage space and lengthy download times so as to address the issue of large ECG files that cannot be opened immediately. It should be noted that, the streaming method 200 of the present disclosure is not limited to implementation through the streaming system 100 of the present disclosure. Each component in the streaming system 100 can be arbitrarily integrated into various combinations to perform the functions of the streaming method 200.

Therefore, the capacity of each data is reduced by segmenting the original data into multiple data, and it is favorable for improving the reading speed of each data. By adding the timestamp to each piece of the data, it is favorable for improving the access accuracy of each piece of the data. By using the webpage cross-platform streaming instead of downloading files, it is favorable for saving device storage space and lengthy download times so as to address the issue of large ECG files that cannot be opened immediately. By editing the data, it is favorable for managers to correct the data and analyze results so as to improve the data accuracy.

### 3rd Embodiment

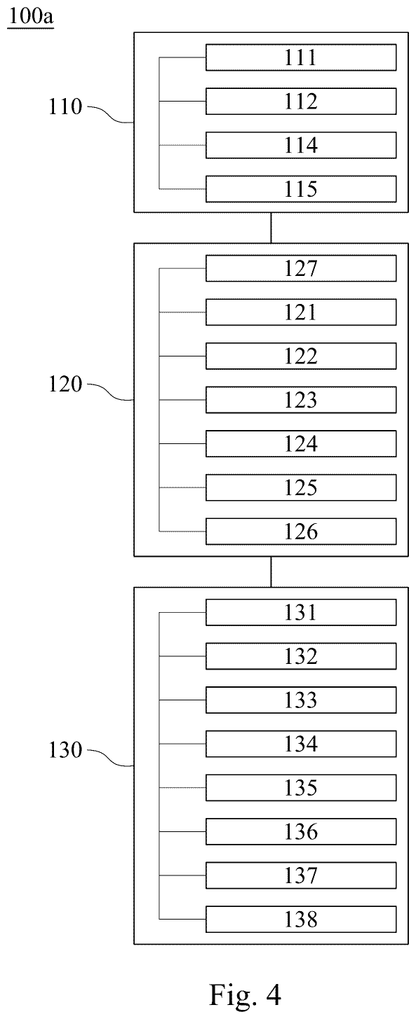

FIG. 4 is a block diagram of a streaming system 100a according to the 3rd embodiment of the present disclosure. As shown in FIG. 4, in the 3rd embodiment, the difference between the streaming system 100a of the 3rd embodiment and the streaming system 100 of the 1st embodiment in FIG. 1 is that, the measurement module 110 can further include an original data compressing unit 114 and an original compressed data transmitting unit 115, and the analysis module 120 can further include an original compressed data decompressing unit 127.

The original data compressing unit 114 is configured to compress the original data obtained by the original data generating unit 111 to generate original compressed data. The original compressed data transmitting unit 115 is configured to transmit the original compressed data to the analysis module 120. The original compressed data decompressing unit 127 is configured to decompress the original compressed data to obtain the original data, so that the original data segmenting unit 121 can subsequently segment the original data into the plurality of original segmented data.

Thus, by compressing the original data by the original data compressing unit 114, it is favorable for speeding up the data transmission from the measurement module 110 to the analysis module 120.

### 4th Embodiment

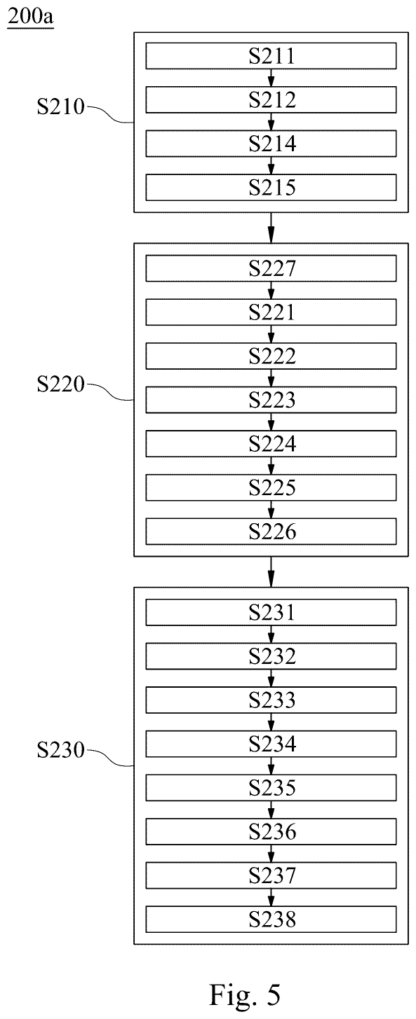

FIG. 5 is a flow chart of a streaming method 200a according to the 4th embodiment of the present disclosure. As shown in FIG. 5, in the 4th embodiment, the difference between the streaming method 200a of the 4th embodiment and the streaming method 200 of the 2nd embodiment in FIG. 3 is that, the measuring step S210 can further include step S214 and step S215, and the analyzing step S220 can further include step S227.

Step S214 and step S215 are sequentially executed after step S212. Step S214 includes compressing the original data to generate the original compressed data. Step S215 includes transmitting the original compressed data. Step S227 is executed before step S221. Step S227 includes decompressing the original compressed data to obtain the original data so as to proceed to step S221 to segment the original data into the plurality of original segmented data.

In detail, the streaming system 100a of the 3rd embodiment of FIG. 4 is configured to implement the streaming method 200a of the 4th embodiment, and is configured to replace downloading files through the webpage cross-platform streaming. It is favorable for saving device storage space and lengthy download times so as to address the issue of large ECG files that cannot be opened immediately. It should be noted that, the streaming method 200a of the present disclosure is not limited to implementation through the streaming system 100a of the present disclosure. Each component in the streaming system 100a can be arbitrarily integrated into various combinations to perform the functions of the streaming method 200a.

Thus, by transmitting the original data after compression, it is favorable for speeding up the data transmission.

Although the present disclosure has been described in considerable detail with reference to certain embodiments thereof, other embodiments are possible. Therefore, the spirit and scope of the appended claims should not be limited to the description of the embodiments contained herein.

It will be apparent to those skilled in the art that various modifications and variations can be made to the structure of the present disclosure without departing from the scope or spirit of the disclosure. In view of the foregoing, it is intended that the present disclosure cover modifications and variations of this disclosure provided they fall within the scope of the following claims.

What is claimed is:

1. A streaming system, which is an electrocardiogram streaming system, comprising a measurement module, an analysis module and a display module;
wherein, the measurement module comprises:
an original data generating unit;
an original data saving unit; and
an original data transmitting unit;
wherein, the analysis module is signally connected to the measurement module and the display module, and the analysis module comprises:
an original data segmenting unit;
an original segmented data timestamp assigning unit;
an original segmented data saving unit;
an original segmented data filtering unit; and
a filtered segmented data processing unit comprising a filtered segmented data compressing subunit;
wherein, the display module comprises:
a data extracting unit comprising a filtered segmented compressed data extracting subunit;
a filtered segmented compressed data decompressing unit;
a webpage cross-platform streaming unit; and
a data presenting unit.

2. The streaming system of claim 1, wherein the filtered segmented data compressing subunit compresses a filtered segmented data using Huffman coding.

3. The streaming system of claim 1, wherein a capacity of a filtered segmented compressed data of the filtered segmented compressed data extracting subunit is less than 50 KB.

4. The streaming system of claim 1, wherein the web design language of the webpage cross-platform streaming unit comprises Hyper Text Markup Language 5 (HTML5).

5. The streaming system of claim 1, wherein the display module further comprises a webpage time limiting unit.

6. The streaming system of claim 1, wherein the display module further comprises a data temporary saving unit.

7. The streaming system of claim 1, wherein the display module further comprises a data applying unit, and the data applying unit comprises a data editing subunit.

8. The streaming system of claim 1, wherein the display module further comprises an URL standardization unit.

9. A streaming system, which is an electrocardiogram streaming system, comprising an analysis module and a display module;
wherein, the analysis module is signally connected to the display module, and the analysis module comprises:
an original data segmenting unit;
an original segmented data timestamp assigning unit;
an original segmented data saving unit;
an original segmented data filtering unit; and
a filtered segmented data processing unit comprising a filtered segmented data compressing subunit;
wherein, the display module comprises:
a data extracting unit comprising a filtered segmented compressed data extracting subunit;
a filtered segmented compressed data decompressing unit;
a webpage cross-platform streaming unit;
a data presenting unit; and
a data applying unit comprising a data editing subunit.

10. The streaming system of claim 9, wherein the display module further comprises a webpage time limiting unit.

11. The streaming system of claim 9, wherein the display module further comprises a read optimizing unit, and the read optimizing unit comprises a memory setting subunit.

12. The streaming system of claim 11, wherein the read optimizing unit further comprises a data reading speed setting subunit.

13. The streaming system of claim 12, wherein the read optimizing unit further comprises a data reading speed displaying subunit.

14. The streaming system of claim 13, wherein the read optimizing unit further comprises a data pre-fetching subunit.

15. The streaming system of claim 9, wherein the display module further comprises a data temporary saving unit.

16. The streaming system of claim 9, wherein the data applying unit further comprises a data comparing subunit.

17. The streaming system of claim 9, wherein the data applying unit further comprises an edited record displaying subunit.

18. The streaming system of claim 9, wherein the data applying unit further comprises a data sharing subunit.

19. The streaming system of claim 9, wherein the data applying unit further comprises a data section downloading subunit.

20. The streaming system of claim 9, wherein the display module further comprises a facilitation unit, and the facilitation unit comprises an interface customization subunit.

21. The streaming system of claim 9, wherein the display module further comprises a facilitation unit, and the facilitation unit comprises a voice subunit.

22. A streaming method, which is an electrocardiogram streaming method, comprising performing a measuring step, an analyzing step and a displaying step;
wherein, the measuring step comprises:
transmitting an original data or an original compressed data;
wherein, the analyzing step comprises:
segmenting the original data into a plurality of original segmented data;
adding a timestamp to each of the original segmented data;
storing the original segmented data;
filtering the original segmented data to generate a filtered segmented data; and
processing the filtered segmented data to generate a filtered segmented compressed data;
wherein, the displaying step comprises:
extracting the filtered segmented compressed data;
decompressing the filtered segmented compressed data;
providing a webpage with cross-platform data streaming capability;
presenting multiple data on the webpage, the data comprising the original data, the original segmented data, the filtered segmented data, or the filtered segmented compressed data; and
editing the data.

23. The streaming method of claim 22, wherein the displaying step further comprises setting a limited time for the webpage.

24. The streaming method of claim 23, wherein the displaying step further comprises storing the data temporarily.

25. The streaming method of claim 24, wherein the displaying step further comprises sharing the data.
